(19) United States
(12) Patent Application Publication
(10) Pub. No.: US 2017/0216110 A1
(43) Pub. Date: Aug. 3, 2017

(54) ABSORBING AND DISTRIBUTING LAYER FOR A LIQUID TO BE ABSORBED AND PRODUCTS PRODUCED THEREFROM

(71) Applicant: Fitesa Germany GmbH, Peine (DE)

(72) Inventors: Helmut Hartl, Braunschweig (DE); Elena Novarino, Hannover (DE); Harald Siebner, Braunschweig (DE)

(21) Appl. No.: 15/329,680

(22) PCT Filed: Jul. 29, 2015
(86) PCT No.: PCT/EP2015/067365

§ 371 Date: Jan. 27, 2017

(30) Foreign Application Priority Data
Aug. 5, 2014 (DE) 10 2014 011 372.8

(51) Int. Cl. A61F 13/538; A61F 13/15; A61L 15/24; A61F 13/537

(52) U.S. Cl. CPC A61F 13/538; A61F 13/5376; A61F 13/15658; A61L 15/24

(57) ABSTRACT

The present invention relates to an absorbing and distributing layer for a liquid to be absorbed, to a sheet material at least including a corresponding absorbing and distributing layer, to a hygiene article at least including a corresponding absorbing and distributing layer, to a method for producing a corresponding sheet material, and to the use of the corresponding absorbing and distributing layer.

The present invention relates to an absorbing and distributing layer for a liquid to be absorbed, to a sheet material at least comprising a corresponding absorbing and distributing layer, to a hygiene article at least comprising a corresponding absorbing and distributing layer, to a method for producing a corresponding sheet material, and to the use of the corresponding absorbing and distributing layer.

Absorbing and distributing layers are suitable for absorbing and distributing liquids, and optionally transporting the liquid to other layers, for example, to an absorbent core. In particular, absorbing and distributing layers are used in hygiene products, such as diapers, incontinence pads, panty liners, sanitary towels, or cosmetic pads. These hygiene products are designed to absorb body fluids and retain them after absorption. In hygiene articles, the absorbing and distributing layers inter alia serve to absorb the often acutely and/or suddenly exiting body fluids as quickly as possible, distribute them and transmit them to other absorption layers, in order that a dry feel can be maintained for the user of the hygiene articles.

Absorbing and distributing layers for liquids are known from the prior art.

U.S. Pat. No. 5,314,743 described absorbing and distributing layers comprising shaped fibers with at least one notch, and serving to absorb and transport liquids. The absorbing and distributing layers described are used as intermediate layers between a fluid-permeable body side pad and a fluid-impermeable outer coat.

However, it is often the case with hygiene articles that a so-called rewet or wetback effect of the hygiene article occurs. “Rewetting” in the context of a hygiene product means that liquid is released again towards the body from the layer loaded with liquid, for example, the absorbent core. The aim of modern hygiene product is that the user's skin remains as dry as possible, i.e., as little liquid as possible is transmitted back towards the body.

Although the majority of the liquid can be retained well in the absorbent body by the use of modern absorbent cores comprising plastics, so-called superabsorbers, that can absorb a multiple of their own weight of liquids, the absorbent body may feel wet.

In addition, in hygiene articles, such as diapers, incontinence pads, panty liners or sanitary towels, a high pressure is often exerted on the absorbent cores, for example, when the person wearing the hygiene article sits or is sitting down, and a major part of the body weight thereby acts on the absorbent core. It is then possible that liquid already absorbed is pressed out of the absorbent core and released towards the body.

As a consequence of the rewetting effect of a hygiene article, irritations of the wearer's skin may occur because of the permanently existing wet microclimate.

In order to enable as comfortable as possible a wearing feel for the wearer of hygienic products, two more properties are particularly important in addition to low rewetting properties. On the one hand, it is important that the upper layer of the hygiene product, which is in contact with the skin, possesses sufficient softness that ensures a pleasant feel on the skin. On the other hand, the hygiene products are to be as small and thin as possible, in order that they are not perceived by the wearer him- or herself or by other persons during the wearing if possible, and/or do not lead to the wearing person being hindered or hampered.

Usually, the skin feel or the softness of the hygiene products is adjusted by a liquid-permeable cover layer, also referred to as the top sheet, which covers the absorbing and distributing layer and is intended for contacting the skin of the wearers. Although a pleasant feel can be ensured by the use of this cover layer, the additional layer has the result that the total thickness of the hygiene product increases, which deteriorates the wearing properties of the hygiene product as compared to thinner hygiene products.

Therefore, there is a constant need for improved hygiene products that are very thin and at least maintain or even improve the wearing feel, the absorbency and the rewet as compared to previously known hygiene products.

The use of absorbing and distributing layers is not limited to the use in hygiene articles, such as cosmetic pads, diapers, incontinence pads, panty liners or sanitary towels. Absorbing and distributing layers with improved properties can be employed for any use where the quick absorption and distribution of liquids is necessary, for example, in wiping cloths, as an insulation material, as a filter material, or as a envelope material for absorbent cores.

Therefore, it is the object of the present invention to provide an absorbing and distributing layer that has at least one, preferably several, of the following properties:
high absorbency for liquids, including under pressure,
high distributing capacity of liquids in the layer,
low rewetting effect,
improved wearing properties, or improved other properties while the wearing properties remain the same,
low production and/or material cost,
low density of the layer,
low thickness of the layer,
low weight of the layer,
low material consumption in the production of the layer.

According to the invention, this object is achieved by a absorbing and distributing layer for a liquid to be absorbed having the features of claim 1, by a sheet material having the features of claim 10, by a hygiene article having the features of claim 11, by a process having the features of claim 12, and by the use according to claim 13. Further aspects of the present invention can be seen from the following description, especially the described Examples, and the enclosed claims and Figures. All combinations or only individual combinations between the following features and aspects of the absorbing and distributing layer can be utilized together.

Further, it is also provided and possible to respectively combine single or several features of the absorbing and distributing layer in any manner.

An absorbing and distributing layer for a liquid to be absorbed is proposed that comprises at least one non-woven consisting of trilobal fibers. Thus, an absorbing and distributing layer according to the invention may consist of a non-woven. It is also possible that it comprises several non-wovens consisting of trilobal fibers according to the invention. Another embodiment provides that the absorbing and distributing layer has one or more layers of non-woven, in which at least one of the layers, preferably at least two layers, consist of trilobal fibers. Preferably, each layer consists of trilobal fibers. In a particularly preferred embodiment, the absorbing and distributing layer according to the invention only has fibers with a trilobal shape. Fibers having a different cross-section are not contained in this embodiment. The fibers may be identical or different, and may be made, especially spun, from the same material, especially thermoplastic material, preferably as a spun-bonded fabric layer. When the absorbing and distributing layer is described in the following, this primarily means the non-woven according to the invention, or in some embodiments the non-wovens according to the invention. If the absorbing and distributing layer according to the invention comprises not only a non-woven according to the invention, but further non-wovens, then the whole layer including all non-wovens is to be understood accordingly.

According to a preferred embodiment of the present invention, an absorbing and distributing layer has such a design that the absorbing and distributing layer has a rewet of from 0.01 g to 0.50 g, preferably from 0.05 g to 0.3 g, more preferably from 0.1 g to 0.25 g, as measured by the EDENA standard test: WSP 80.10 (05).

This avoids that liquid already absorbed and optionally transmitted by the absorbing and distributing layer is released again when pressure is exerted on the absorbing and distributing layer. In particular, when the absorbing and distributing layer is employed in hygiene products, a pleasant wear feel for the wearer can be achieved thereby, and a wet microclimate, which may lead to irritations of the wearer's skin, is avoided.

In addition, it is preferred that the absorbing and distributing layer is hydrophilic in nature.

The term “hydrophilic” designates a material that has a water-in-air contact angle of less than 90 degrees. The water-in-air contact angle is determined according to the description in the book “Absorbency”, edited by P. K. Chatterjee (Elsevier, New York, 1985). The hydrophilicity of the absorbing and distributing layer can be adjusted in one of different ways. For example, inherently hydrophilic trilobal fibers may be used. “Inherently” hydrophilic fibers means fibers that are hydrophilic but free of surface modifications or treatments, for example, free of surface-active agents, spinning additives, glazing agents etc. Also, it is possible to adjust the hydrophilicity of the fibers by corresponding surface modifications or treatments.

Therefore, in another embodiment, it is preferred that the absorbing and distributing layer is provided with a hydrophilic finish by a finishing process familiar to the skilled person, for example, soaking, spraying, padding or kiss-roll application. In particular, absorbing and distributing layers that have been treated with a surfactant solution are preferred. In principle, all kinds of surfactants are suitable, i.e., anionic, cationic and also non-ionic or zwitterionic surfactants. For example, the surfactant solution may be a solution comprising Stantex® from the company Cognis, Düsseldorf (Germany), or Silastol PHP26 from Schill & Seilacher, Böblingen (Germany), and/or a solution containing castor ethoxylates and/or PEG diesters.

According to a preferred embodiment of the present invention, the absorbing and distributing layer has a strike-through time of less than 4 seconds as measured by the EDENA standard test: WSP 70.3 (05).

Absorbing and distributing layers having a short strike-through time have the advantage that liquids can be absorbed and transmitted quickly. In particular, when the absorbing and distributing layers are used in hygiene products, such as diapers, it is important that suddenly occurring amounts of liquid, as are produced during urination, for example, are absorbed and transmitted as quickly as possible. For example, the wearing comfort can be improved thereby, and lateral leaking of the liquid can be avoided.

Usually, the strike-through time and the rewet are opposite properties. Layers that absorb liquids quickly usually also transmit them quickly and lead to a high rewet, especially if pressure is exerted on the layer. In the present case, it has been surprisingly found that the absorbing and distributing layers absorb liquids very well and have a short strike-through time, but also have a very low rewet at the same time, because a return of the liquid is prevented. This property can be explained by the shape of the trilobal fibers. As soon as pressure is exerted on the absorbing and distributing layers and thus on the individual fibers as well, the fibers are presumably pressed flat and thus become broader, which results in a lower rewet. This behavior cannot be achieved with usual round fibers.

In a preferred embodiment of the absorbing and distributing layer, the absorbing and distributing layer has such a design that the trilobal fibers of the absorbing and distributing layer predominantly consist of a polyolefin, especially polypropylene or polyethylene, copolymers or mixtures thereof. In addition to a polymer, fibers usually contain pigments, stabilizers, especially against thermal chain degradation, and optionally other batches. However, their components usually comprise less than 5% by weight of the fibers, except when fillers are used. The content of fillers may absolutely be higher. Thermoplastic polymers that may also be employed include, for example, polyethylene terephthalate, polytrimethylene terephthalate, polylactides, copolymers or mixtures thereof. Even more preferred is the use of polypropylene in the form of Ziegler-Natta PP or metallocene PP, but also in the form of mixtures thereof.

When polypropylene is used as the fibrous material, the use of isotactic polypropylene is preferred.

For example, by using the above stated materials, the surface quality of the absorbing and distributing layer can be adjusted so that the absorbing and distributing layer has sufficient softness, and a pleasant feel on the skin is ensured.

Usually, the absorbing and distributing layer, in particular, is stabilized by air-through bonding or resin bonding. These bonding methods provide the non-wovens with some dimensional stability, but also with a high stiffness, which can act against the wearing comfort depending on the extent thereof, and on the intended use. Mainly for air-through bonding, the use of bicomponent fibers, i.e., a low melting outer shell with a thermally stable fiber core, which may be expensive, is often prescribed. The air-through bonding method is applied, in particular, for “loosening” the round fibers, which are densely packed in the non-woven. Thus, the required porosity of the non-woven is generated in order to ensure as uniform as possible a distribution of the penetrating liquid. On the other hand, this improves the per se low resistive force or crease recovery, also referred to as “resilience”, of non-wovens made of round fibers. The bonding method that is as such most favorable economically, i.e., thermal calendaring, has some drawbacks here. The fibers, which are already densely packed by themselves, would be compacted even further between heated rollers. However, this would completely destroy the required non-woven properties.

Further, it has surprisingly been found that non-wovens made of trilobal fibers can absolutely be thermally calendared without the resulting non-wovens in the least losing the required functionality, because of their fiber cross-section, particularly if based on polypropylene and/or polyethylene. This is an extraordinarily desirable finding in economic terms because better non-woven functions can now be prepared more cheaply and more quickly.

Depending on the intended use of the absorbing and distributing layer, different masses per unit area of the absorbing and distributing layer are reasonable. For hygiene articles, thin and/or lightweight absorbing and distributing layers are preferred, because the wearing comfort for the wearer can be improved thereby. However, when absorbing and distributing layer are used in cloths or cosmetic pads, for example, it may also be preferable that the absorbing and distributing layer is thicker or heavier, because the liquid uptake of the absorbing and distributing layer can usually be increased thereby.

According to a preferred embodiment of the present invention, an absorbing and distributing layer has a mass per unit area of from 5 g/m² to 60 g/m², preferably from 7 g/m² to 40 g/m², more preferably from 8 g/m² to 20 g/m², especially from 10 g/m² to 17 g/m², according to DIN EN 29073-1.

It has been found that absorbing and distributing layers having the above mentioned masses per unit area have particularly good wearing properties while showing a very good rewet behavior.

According to a particularly preferred embodiment of the present invention, an absorbing and distributing layer has such a design that the absorbing and distributing layer has a rewet of from 0.1 g to 0.25 g, as measured according to the EDENA Standard Test: WSP 80.10 (05), and a mass per unit area of from 10 g/m² to 15 g/m² according to DIN EN 29073-1.

According to a preferred embodiment of the present invention, the absorbing and distributing layer comprises several embossing surfaces.

The embossing surfaces may be trilobal, round, especially oval, elliptic or circular, polygonal, especially rectangular, square, triangular, quadrangular, pentagonal or hexagonal, star-shaped or designed in the form of patterns. Also, lands may be employed, straight and/or curved lands. Also, different embossing surfaces with different geometries may be combined on one absorbing and distributing layer.

The embossing surfaces can improve the mechanical, haptic and optical properties of the absorbing and distributing layer.

In addition, properties such as the rewet properties and the mechanical properties can be controlled by the arrangement and size of the embossing surfaces.

Preferred are absorbing and distributing layers in which each of the embossing surfaces has a surface area of from 0.5 mm² to 5 mm², preferably from 2 mm² to 4 mm².

In our own examinations, it has been found that good rewet properties can be obtained thereby.

According to a preferred embodiment of the present invention, the absorbing and distributing layer has such a design that the absorbing and distributing layer has a cumulated surface area of the embossing surfaces of 3% to 35%, preferably 5% to 30%, more preferably 10% to 25%, of the total surface area of the absorbing and distributing layer.

In a preferred embodiment, the embossing surfaces have an elliptic design and have a length of the major axis of about 2.3 mm to 2.7 mm, preferably 2.45 mm, and a length of the minor axis of about 1.9 mm to 2.3 mm, preferably 2.08 mm, the surface area of the embossing surface is about 3.8 mm² to 4.3 mm², preferably 4 mm², and the cumulated surface area of the embossing surfaces on the absorbing and distributing layer is from about 23% to 27%, preferably 25%. Preferably, several embossing surfaces are arranged around another one in the middle of a thus formed pattern. For example, six embossing surfaces are arranged hexagonally, and another embossing surface is located in the center of the hexagon.

In another preferred embodiment, the embossing surfaces have trilobal and/or rod-shaped designs. For example, the surface area of the embossing surface is about 0.850 mm² to about 1.150 mm², preferably 0.987 mm², for a trilobal design, and about 0.550 mm² to about 0.780 mm², preferably 0.655 mm², for a rod-shaped design, and the cumulated surface area of the embossing surfaces on the absorbing and distributing layer is preferably from 15% to 17.5%, preferably 16.2%, for a mixture of trilobal and rod-shaped embossing surfaces. For example, the embossing surfaces are arranged on the absorbing and distributing layer in such a way that a hexagonal pattern is formed, or another uniformly repeated pattern of embossing surfaces in an approximately circular arrangement is formed.

In another preferred embodiment, the embossing surfaces have a circular design. The diameter of the embossing surface is about 0.875 mm to about 1.155 mm², preferably 1 mm². The surface area of the embossing surface is preferably from 0.766 mm² to 1.334 mm², preferably 0.785 mm², and the cumulated surface area of the embossing surfaces on the absorbing and distributing layer is preferably from 16.5% to about 18.3%, preferably 17.02%. For example, the embossing surfaces are arranged on the absorbing and distributing layer in such a way that a hexagonal pattern is formed, 12 embossing patterns for each hexagon being arranged (1 embossing surface per corner of the hexagon and one embossing surface each between two corners). However, other patterns are also possible.

In another preferred embodiment, the embossing surfaces have a rod-shaped design. The thickness of the rod is, for example, from 0.4 mm to 0.7 mm, preferably 0.5 mm, and the length of the rod is about from 1.9 mm to 3.2 mm, preferably about 2.4 mm. The surface area of the embossing surface is preferably from about 0.76 mm² to 2.24 mm², especially about 1.146 mm². A cumulated surface area of the embossing surfaces on the absorbing and distributing layer is preferably from 9.5% to about 13.5%, preferably about 10.2%.

A preferred embodiment of the present invention relates to an absorbing and distributing layer having trilobal fibers with a titer of 1 dtex to 10 dtex as determined with a microscope, for example, preferably with a titer of 2 dtex to 8 dtex, more preferably with a titer of 2 dtex to 5 dtex.

Also preferred is an absorbing and distributing layer whose trilobal fibers have an organic or inorganic filler.

By using organic or inorganic fillers in the trilobal fibers, various positive effects can be achieved. For example, using less costly fillers can reduce the consumption of more expensive materials, and the price of the fibers can be reduced accordingly. However, the use of fillers and the geometry of the fillers employed may also have effects on the haptic or mechanical properties, for example.

Further preferred are absorbing and distributing layers whose trilobal fibers contain pigments, especially TiO₂.

The use of pigments in the fibers has the effect that the fibers are optically more appealing and also have a higher opacity. In particular for thin absorbing and distributing layers, it is preferred that other possible layers that are below the absorbing and distributing layer do not shine through. However, the use of corresponding pigments may also improve the aesthetic appearance of the absorbing and distributing layer, especially when the absorbing and distributing layer has absorbed colored liquids.

In our own examinations, it has been found that absorbing and distributing layers are particularly suitable if the absorbing and distributing layer includes filaments in the machine direction (MD) and filaments transverse to the machine direction (CD), and the ratio of filaments in the machine direction (MD) to filaments transverse to the machine direction (CD) is 1.1 to 5.0.

In addition, it is preferred that the absorbing and distributing layer comprises or consists of a spunbond layer.

Preferred are absorbing and distributing layers that comprise a spunbond layer consisting of trilobal polypropylene fibers.

These absorbing and distributing layers can be calendared, and the cost-intensive methods, such as “air-through bonding” or “resin bonding”, can be avoided.

Further, it may be advantageous if the absorbing and distributing layer additionally includes a melt-blown layer.

In this case, it is particularly preferred if the melt-blown layer is provided between two spunbond layers.

According to one embodiment, absorbing and distributing layers that are biologically degradable are proposed.

An absorbing and distributing layer is understood to be biologically degradable if it is degraded to at least 50% by weight within 12 weeks in industrial composting according to European standard EN 13432.

Also preferred is an absorbing and distributing layer whose trilobal fibers have an arm thickness of 4 to 10 μm, preferably an arm thickness of 5 to 9 μm, especially an arm thickness of 5 to 8 μm, and/or have an arm length of 10 to 40 μm, preferably an arm length of 12 to 30 μm, more preferably an arm length of 14 to 25 μm.

The arm thickness and the arm length of the trilobal fibers are determined by means of a microscope. Thus, the trilobal fibers are embedded in a suitable synthetic resin and subsequently sliced transversely to the fiber length. The arm length is measured from the center of the trilobal fiber to the tip of the arm. The arm thickness is determined at mid-length of the arm.

Surprisingly, it has been found that fibers having the described arm lengths and/or arm thicknesses have particularly good properties in terms of liquid transmission and rewet. In particular, at shorter arm lengths and/or larger arm thicknesses, the advantageous low rewet, as surprisingly found, is not obtained. In addition, the production of non-wovens as used in the absorbing and distributing layer according to the invention is complicated if the arm length, in particular, deviates from the mentioned values.

Therefore, a non-woven of an absorbing and distributing layer preferably consists of trilobal fibers in which the ratio of arm thickness to arm length is within a range of from 1:10 to 1:1, especially from 1:8 to 1:1.5, preferably from 1:5 to 1:2, especially from 1:5 to 1:2.5, for example, being 1:5, 1:4, 1:3.5, or 1:3. Surprisingly, it has been found that a good effect in terms of rewet can be achieved here, especially within a range of from 1:10 to 1:2.

In particular, the trilobal fibers are melt-spun fibers prepared using spinning plates. The spinning plates preferably have trilobal bores that have an arm thickness of 50 to 300 μm, preferably an arm thickness of 100 to 250 μm, especially an arm thickness of 150 to 230 μm, and/or have an arm length of 400 to 900 μm, preferably an arm length of 500 to 800 μm, more preferably an arm length of 520 to 760 μm.

It has been found that trilobal fibers can be prepared particularly well in this way. In particular, trilobal fibers for preparing the layers according to the invention can be prepared at a ratio of arm thickness to arm length of the spinning plates within a range of from 1:10 to 1:1, especially from 1:8 to 1:1.5, preferably from 1:5 to 1:2, especially from 1:5 to 1:2.5, for example, being 1:5, 1:4, 1:3.5, or 1:3. Surprisingly, it has been found that particularly dimensionally stable trilobal fibers can be prepared here, especially within a range of from 1:10 to 1:2. In this context, “dimensionally stable” means that the fibers obtained have the same or approximately the same ratio of arm thickness to arm length as the spinning plates.

In this connection, the arm thickness is particularly critical. If the spinning plates have a significantly thinner arm thickness, high shear rates, inter alia, will occur during the fiber production, so that the material expands after the passage through the spinning plate. This can lead to unstable spinning. Also, sufficient control of the final fiber geometry is no longer ensured.

In contrast, if the spinning plates have too thick an arm thickness as compared to the arm length, a trilobal cross-section of the fiber can no longer be achieved. Rather, such a fiber then has a triangular cross-section, which results in a completely different physical behavior of the resulting non-woven material.

In addition, at the ratios according to the invention, fibers can be prepared whose arm thickness and arm length are approximately constant over the length of the entire fiber, which leads to a good property of the absorbing and distributing layer according to the invention in terms of absorbency, wear feel, but also rewet. Thus, for example, a trilobal fiber cross-section that is not essentially constant can have the result that this advantageous behavior partially can no longer be ensured. This can just be avoided by the ratios preferred according to the invention during the production and thus also in the fibers.

Also preferred is an absorbing and distributing layer that has an air permeability according to DIN EN ISO 9237 (12-1995) (surface area of the measuring head: 20 cm², test pressure: 200 Pa) of from 4000 to 9500 L/m²s, preferably from 5000 to 9000 L/m²s.

In addition, it is preferred that the absorbing and distributing layer has a thickness of from 0.3 to 2.0 mm, preferably a thickness of from 0.3 to 1.5 mm, more preferably a thickness of from 0.4 to 1.0 mm, even more preferably from 0.4 to 0.6 mm.

The embodiments of the absorbing and distributing layer as stated in this description can be arbitrarily combined with one another. The skilled person can adjust the desired properties of the absorbing and distributing layer in test experiments by varying the individual parameters.

In our own experiments, it has been found that, in particular, an absorbing and distributing layer for a liquid to be absorbed is preferred that consist of a non-woven at least comprising, preferably consisting of, trilobal fibers, wherein the absorbing and distributing layer has a rewet as measured by the EDENA standard test WSP 80.10 (05) of from 0.1 g to 0.25 g, the absorbing and distributing layer is hydrophilic, wherein the trilobal fibers of the absorbing and distributing layer are made of polypropylene, preferably isotactic polypropylene, wherein the absorbing and distributing layer has a mass per unit area according to DIN EN 29073-1 of from 10 g/m² to 17 g/m², wherein the absorbing and distributing layer comprises several embossing surfaces having a surface area of from 2 mm² to 5 mm² each, and the absorbing and distributing layer has a cumulated surface area of the embossing surfaces of 3% to 32% of the total surface area of the absorbing and distributing layer, the trilobal fibers have a titer of 3 dtex to 6 dtex, wherein the absorbing and distributing layer has, for example, two spunbond layers and one melt-blown layer, wherein the melt-blown layer is provided between the two spunbond layers, and wherein the absorbing and distributing layer has a thickness of from 0.4 mm to 0.6 mm.

In addition, it is particularly preferred that the non-woven of the absorbing and distributing layer completely consists of trilobal fibers, and the absorbing and distributing layer has a lower rewet by a factor of 1:2, preferably by a factor of 1:5, more preferably by a factor of 1:8, as compared to an absorbing and distributing layer produced under identical conditions, but whose non-woven completely consists of round fibers with the same titer as the trilobal fibers. Thus, if an absorbing and distributing layer whose non-woven completely consists of round fibers has a rewet of, for example, 2 g, then the rewet of an absorbing and distributing layer whose non-woven completely consists of trilobal fibers should be lower than 1 g (factor 1:2), preferably lower than 0.4 g (factor 1:5), more preferably lower than 0.25 g (factor 1:8).

In particular, it is preferred that both kinds of absorbing and distributing layers that are compared with one another:
have identical embossing surfaces, preferably as stated above as preferred with respect to the surface area of the embossing surfaces, the geometry of the embossing surfaces, and the cumulated surface area of the embossing surfaces;
have identical masses per unit area, preferably as stated above as preferred;
have identical thicknesses of the absorbing and distributing layer, preferably as stated above as preferred;
   and the fibers are prepared from identical materials, preferably as stated above as preferred, wherein all the values and properties are determined under identical conditions.

Another aspect in connection with the present invention relates to a sheet material at least consisting of:
an absorbing and distributing layer as described above; and
an absorption layer.

Preferred is a sheet material that additionally comprises a back sheet, wherein the absorption layer is provided between the back sheet and the absorbing and distributing layer.

Further preferred is a sheet material that does not bear any further layer on the absorbing and distributing layer. For example, a top sheet, or its function, may be part of the absorbing and distributing layer.

Another aspect of the present invention relates to a hygiene article at least consisting of an absorbing and distributing layer as proposed above.

Preferred is a hygiene article in which said hygiene article is an article selected from the group consisting of diapers, incontinence pads, panty liners, sanitary towels, and cosmetic pads.

Further preferred is a hygiene article in which the proposed absorbing and distributing layer is provided in such a way that it has direct contact with the skin when used as usual, i.e., does not have a further cover layer, for example, in the form of a top sheet.

Since the absorbing and distributing layer is also suitable for having direct contact with the skin because of its very good mechanical, optical and haptic properties, an additional layer as a cover layer or cover sheet, for example, a top sheet, can be dispensed with. Thus, it is possible that either the thickness of the hygiene article is reduced, or for the same thickness, the efficiency of the hygiene article is improved, for example, as compared with hygiene articles that additionally have a cover layer on top of the absorbing and distributing layer.

However, saving an additional layer can also simplify the production process of hygiene articles, thus also reducing the production cost and product cost. In addition, the pack size of the finished hygiene articles may also be reduced.

In another embodiment, the object of the present invention is achieved by the use of trilobal fibers in an absorbing and distributing layer for reducing or preventing rewet. Surprisingly, it has been found that absorbing and distributing layers absorb liquid very well and have a short strike-through time, but also have a low rewet at the same time, if they comprise, especially consist of, trilobal fibers. The use of the trilobal fibers enables a return of the liquid to be prevented. This behavior cannot be achieved with usual round fibers as described in the prior art, and was not to be expected in this way either.

A preferred aspect of the present invention relates to a process for producing a sheet material, comprising the following steps:
providing an absorbing and distributing layer as described above;
contacting it with an absorption layer.

Further aspects in connection with the present invention relate to advantageous uses of the absorbing and distributing layer described for the preparation of:
hygiene articles, especially diapers, incontinence pads, panty liners, sanitary towels, cosmetic pads;
cleaning cloths, wiping cloths, mop cloths;
filters, for example, for gases, aerosols, and liquids;
wound dressings, wound compress pads;
insulation materials, sound-absorbing non-woven materials;
interlinings;
roof lining membranes;
geotextiles; or
covers for field crop and vegetables production.

Further advantageous embodiments are shown in the following Figures. However, the respective features seen therefrom are not limited to individual Figures or embodiments. Rather, one or more features of the above description can be combined in addition to further embodiments.

Herein:

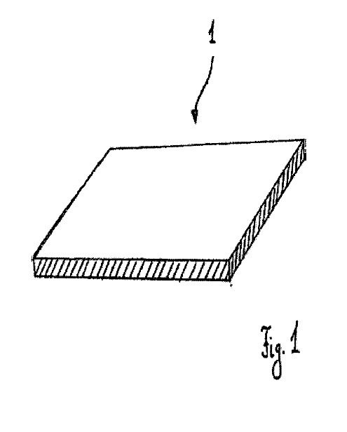

FIG. 1 shows a schematic representation of an absorbing and distributing layer;

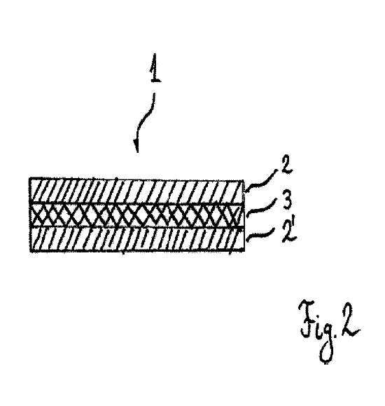

FIG. 2 shows a schematic representation of an absorbing and distributing layer having a melt-blown layer and two spunbond layers;

FIG. 3a shows a schematic representation of an absorbing and distributing layer;

FIG. 3b shows a schematic representation of an absorbing and distributing layer under the action of a pressure;

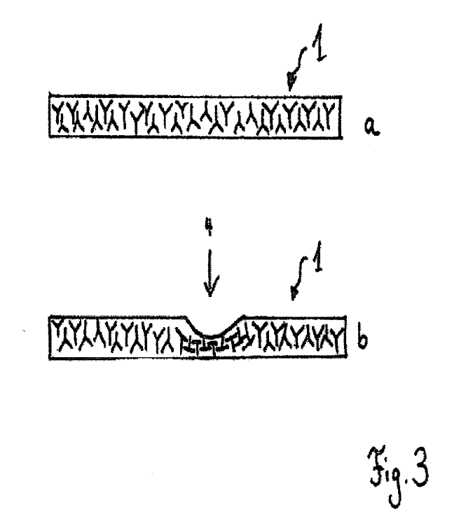

FIG. 4 shows a schematic representation of a sheet material with an absorbing and distributing layer and an absorption layer;

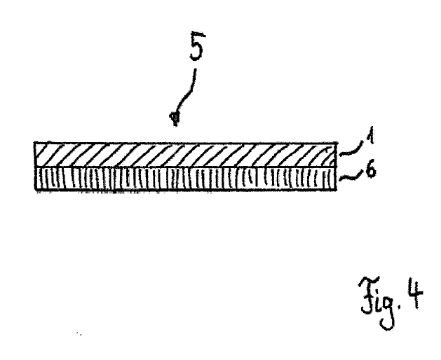

FIG. 5 shows a schematic representation of a sheet material with an absorbing and distributing layer and an absorption layer under the action of water;

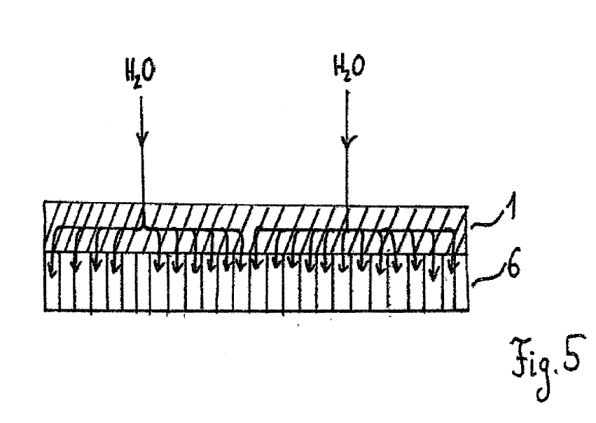

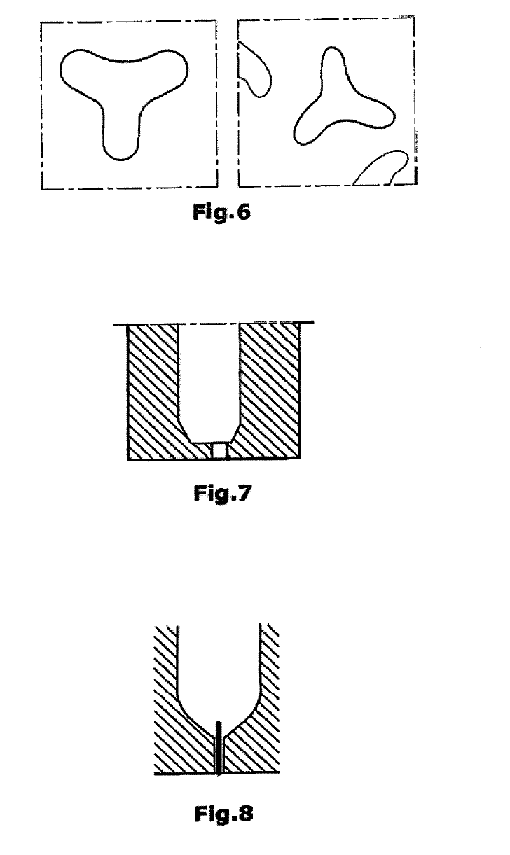

FIG. 6 shows optical micrographs of trilobal fibers;

FIG. 7 shows a schematic structure of a nozzle for producing trilobal fibers;

FIG. 8 shows a schematic structure of a nozzle for producing trilobal fibers;

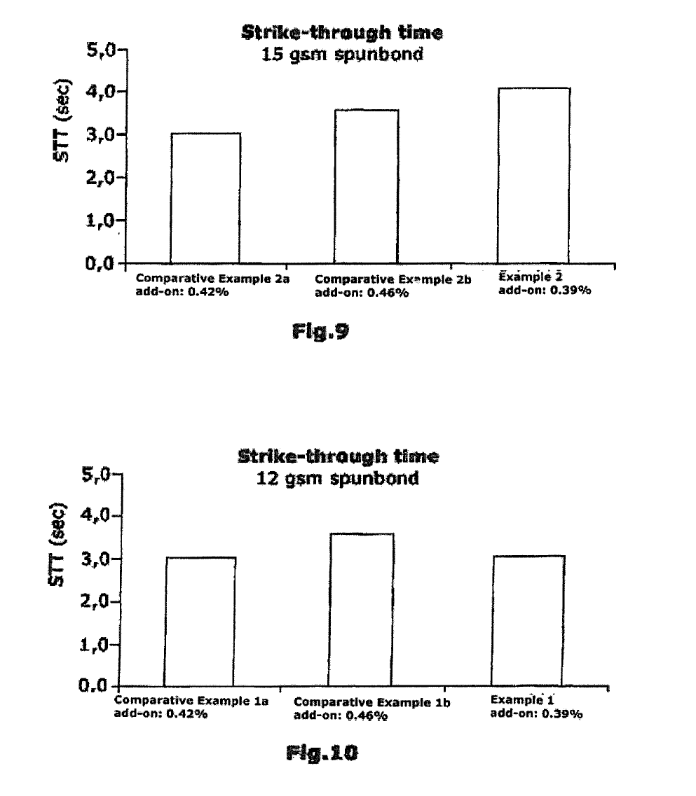

FIG. 9 shows a graphical representation of the strike-through times (SST) determined in the Examples for Comparative Examples 2a and 2b and for Example 2;

FIG. 10 shows a graphical representation of the strike-through times (SST) determined in the Examples for Comparative Examples 1a and 1b and for Example 1;

FIG. 11 shows a graphical representation of the rewet values determined in the Examples for Comparative Examples 2a and 2b and for Example 2; and

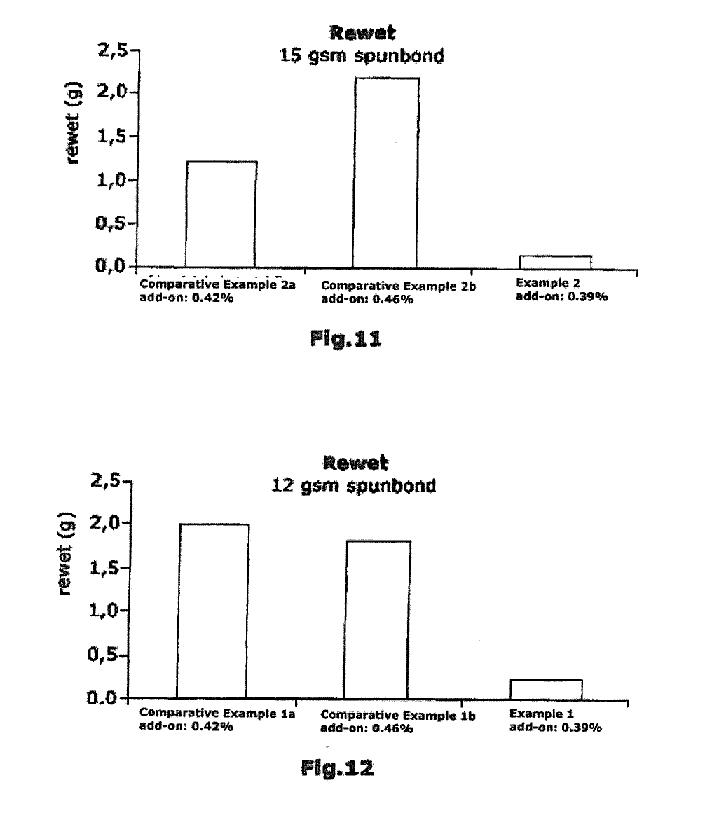

FIG. 12 shows a graphical representation of the rewet values determined in the Examples for Comparative Examples 1a and 1b and for Example 1.

FIG. 1 schematically shows a part of an absorbing and distributing layer 1 as described above.

FIG. 2 schematically shows the structure of an absorbing and distributing layer 1 as described above, having a melt-blown layer 3 and two spunbond layers, wherein the melt-blown layer 3 is provided between the two spunbond layers 2, 2′. The whole assembly represents an absorbing and distributing layer 1 as described above.

FIG. 3a schematically represents an absorbing and distributing layer 1 as described above. FIG. 3b schematically represents an absorbing and distributing layer 1 as described above under the action of a pressure 4. The trilobal fibers of the absorbing and distributing layer 1 as described above are deformed and compressed under the action of pressure 4, forming a compact layer.

FIG. 4 schematically represents a sheet material 5 as described above comprising an absorbing and distributing layer 1 as described above, and an absorption layer 6.

The illustration in FIG. 5 schematically represents a sheet material 5 with an absorbing and distributing layer 1 and an absorption layer 6 under the action of a liquid, such as water, H₂O. The water H₂O is absorbed by the absorbing and distributing layer 1 and distributed in the absorbing and distributing layer 1, before it is subsequently transmitted to the absorption layer 6.

FIG. 6 shows optical micrographs of trilobal fibers of an absorbing and distributing layer 1.

FIG. 7 schematically shows an exemplary structure of a nozzle for producing trilobal fibers.

FIG. 8 schematically shows an exemplary structure of a nozzle for producing trilobal fibers.

FIG. 9 shows the strike-through times (SST) determined in the Examples for Comparative Examples 2a and 2b and for Example 2 in a bar diagram. The non-wovens (spunbond) have a mass per unit area of 15 g/cm².

FIG. 10 shows the strike-through times (SST) determined in the Examples for Comparative Examples 1a and 1b and for Example 1 in a bar diagram. The non-wovens (spunbond) have a mass per unit area of 12 g/cm².

FIG. 11 shows the rewet values determined in the Examples for Comparative Examples 2a and 2b and for Example 2 in a bar diagram. The non-wovens (spunbond) have a mass per unit area of 15 g/cm².

FIG. 12 shows the rewet values determined in the Examples for Comparative Examples 1a and 1b and for Example 1 in a bar diagram. The non-wovens (spunbond) have a mass per unit area of 12 g/cm².

### Measuring Methods

All of the following determinations were performed at 23° C., 1013 mbar and 50% relative humidity, unless otherwise stated or appropriate. The samples were stored for 24 hours under laboratory conditions (23° C. and 50% relative humidity) before being measured.

### Determination of the Filament Titer

The determination of the filament titer is effected by means of a microscope. The conversion of the measured titer (in micrometers) to decitex is performed according to the following formula (PP density=0.91 g/cm³):

$${( \frac{Titer \text{?}}{2} )}^{2} \cdot π \cdot ρ [ \frac{g}{{cm}^{3}} ] \cdot 0.01 = Titer \text{?} [ \frac{g}{{10}^{4} m} ]$$

$$\text{?} \text{indicates text missing or illegible when filed}$$

### Determination of the Mass Per Unit Area

The determination of the mass per unit area is effected on test specimens having a size of 10×10 cm according to DIN EN 29073-1. The thickness of the non-woven material is measured as the distance of two plane-parallel measuring surfaces of a particular size, between which the non-woven materials are under a defined measuring pressure. The method is performed by analogy with DIN EN ISO 9073-2. Load: 125 g; measuring surface area: 25 cm²; measuring pressure: 5 g/cm².

### Determination of Air Permeability

The measurement of the air permeability is effected according to DIN EN ISO 9237. The surface area of the measuring head is 20 cm², the test pressure applied is 200 Pa.

### Determination of Strike-Through Time

The measurement of the strike-through times of the non-woven materials (“liquid strike-through time”) is effected according to the EDENA standard test: WSP 70.3 (05) (“Standard Test Method for Nonwoven Coverstock Liquid Strike-Through Time Using Simulated Urine”).

### Rewet (or Wetback)

The measurement of the rewet of the non-woven materials (“liquid strike-through time”) is effected according to the EDENA standard test: WSP 80.10 (05) (“Standard Test Method for Nonwovens Coverstock Wetback”).

### EXAMPLE 1

A non-woven material with a mass per unit area of 12 g/m² and a titer of 2 dtex was prepared, wherein the fibers have a trilobal structure. The fibers were made of a commercially available Ziegler-Natta polypropylene. The add-on of the non-woven was subsequently adjusted to about 0.4% (determined by extraction with isopropanol; accuracy +/−0.03%). The rewet and the strike-through time of the non-woven prepared were subsequently determined.

### EXAMPLE 2

By analogy with Example 1, a non-woven material with a mass per unit area of 15 g/m² and a titer of 2 dtex was prepared, wherein the fibers have a trilobal structure. The fibers were made of a commercially available Ziegler-Natta polypropylene. The add-on of the non-woven was subsequently adjusted to about 0.4% (determined by extraction with isopropanol; accuracy +/−0.03%). The rewet and the strike-through time of the non-woven prepared were subsequently determined.

### COMPARATIVE EXAMPLE 1a

By analogy with Example 1, a non-woven material with a mass per unit area of 12 g/m² and a titer of 2 dtex was prepared, wherein the fibers have a round structure. The fibers were made of a commercially available Ziegler-Natta polypropylene. The add-on of the non-woven was subsequently adjusted to about 0.4% (determined by extraction with isopropanol; accuracy +/−0.03%). The rewet and the strike-through time of the non-woven prepared were subsequently determined.

### COMPARATIVE EXAMPLE 1b

By analogy with Example 1, a non-woven material with a mass per unit area of 12 g/m² and a titer of 2 dtex was prepared, wherein the fibers have a round structure. The fibers were made of a commercially available metallocene polypropylene. The add-on of the non-woven was subsequently adjusted to about 0.4% (determined by extraction with isopropanol; accuracy +/−0.03%). The rewet and the strike-through time of the non-woven prepared were subsequently determined.

### COMPARATIVE EXAMPLE 2a

By analogy with Example 1, a non-woven material with a mass per unit area of 15 g/m² and a titer of 2 dtex was prepared, wherein the fibers have a round structure. The fibers were made of a commercially available Ziegler-Natta polypropylene. The add-on of the non-woven was subsequently adjusted to about 0.4% (determined by extraction with isopropanol; accuracy +/−0.03%). The rewet and the strike-through time of the non-woven prepared were subsequently determined.

### COMPARATIVE EXAMPLE 2b

By analogy with Example 1, a non-woven material with a mass per unit area of 15 g/m² and a titer of 2 dtex was prepared, wherein the fibers have a round structure. The fibers were made of a commercially available metallocene polypropylene. The add-on of the non-woven was subsequently adjusted to about 0.4% (determined by extraction with isopropanol; accuracy +/−0.03%). The rewet and the strike-through time of the non-woven prepared were subsequently determined.

In the following, the determined values of rewet and of the strike-through time as well as the adjusted add-on values for the Examples and Comparative Examples are represented in tabular form:

| | Strike-through time [s] | Rewet [g] | Add-on |
|---|---|---|---|
| Example 1 | 3.02 ± 0.13 | 0.20 ± 0.10 | 0.39% |
| Example 2 | 4.04 ± 1.00 | 0.13 ± 0.03 | 0.39% |
| Comparative Example 1a | 3.09 ± 0.33 | 2.00 ± 0.93 | 0.42% |
| Comparative Example 1b | 3.53 ± 0.72 | 1.80 ± 0.44 | 0.46% |
| Comparative Example 2a | 3.01 ± 0.29 | 1.21 ± 0.26 | 0.42% |
| Comparative Example 2b | 3.57 ± 0.28 | 2.30 ± 0.99 | 0.46% |

In Examples 1 and 2, it is clearly seen that the rewet is many times lower than that of Comparative Examples 1a to 2b. For illustration, the results have been graphically represented in FIGS. 9 to 12.

1. An absorbing and distributing layer for a liquid to be absorbed,
comprising at least one non-woven consisting of trilobal fibers.

2. The absorbing and distributing layer according to claim 1, wherein said absorbing and distributing layer has a rewet as measured by the EDENA standard test WSP 80.10 (05) of from 0.01 g to 0.50 g, and/or the trilobal fibers of the absorbing and distributing layer comprise a polymer selected from the group consisting of a polyolefin, polyethylene terephthalate, polytrimethylene terephthalate, polylactides, and copolymers or mixtures thereof.

3. The absorbing and distributing layer according to claim 1, wherein said trilobal fibers have an arm thickness of 4 μm to 10 μm.

4. The absorbing and distributing layer according to claim 1, wherein a ratio of arm thickness to arm length is from 1:10 to 1:1.

5. The absorbing and distributing layer according to claim 1, wherein said absorbing and distributing layer comprises several embossing surfaces.

6. The absorbing and distributing layer according to claim 5, wherein each of the embossing surfaces has a surface area of from 0.5 mm² to 5 mm², and the absorbing and distributing layer has a cumulated surface area of the embossing surfaces of 3% to 35%, of the total surface area of the absorbing and distributing layer.

7. The absorbing and distributing layer according to claim 1, wherein said absorbing and distributing layer comprises two spunbond layers and one melt-blown layer, the melt-blown layer being provided between the two spunbond layers.

8. The absorbing and distributing layer according to claim 1, wherein said absorbing and distributing layer has a thickness of from 0.3 to 2.0 mm.

9. The absorbing and distributing layer according to claim 1, wherein said non-woven completely consists of trilobal fibers, and the absorbing and distributing layer has a lower rewet as compared to an absorbing and distributing layer produced under identical conditions, but whose non-woven completely consists of round fibers with the same titer as the trilobal fibers.

10. A sheet material comprising at least:
an absorbing and distributing layer according to claim 1; and
an absorption layer.

11. A hygiene article comprising an absorbing and distributing layer according to claim 1.

12. A process for producing a sheet material according to claim 10, comprising the following steps:
providing an absorbing and distributing layer according to claim 1; followed by
contacting it with an absorption layer.

13. A method of reducing or preventing rewet comprising using trilobal fibers in an absorbing and distributing layer for reducing or preventing rewet.

14. The absorbing and distributing layer according to claim 1, wherein said absorbing and distributing layer has a rewet as measured by the EDENA standard test WSP 80.10 (05) of from 0.05 g to 0.3 g, and the trilobal fibers of the absorbing and distributing layer comprise polypropylene or polyethylene, copolymers or mixtures thereof.

15. The absorbing and distributing layer according to claim 5, wherein each of the embossing surfaces has a surface area of from 2 mm² to 4 mm², and the absorbing and distributing layer has a cumulated surface area of the embossing surfaces of 10% to 25%, of the total surface area of the absorbing and distributing layer.

16. The absorbing and distributing layer according to claim 1, wherein said trilobal fibers have an arm thickness of 12 μm to 30 μm.

17. The absorbing and distributing layer according to claim 1, wherein said absorbing and distributing layer has a thickness of from from 0.4 to 0.6 mm.
